(19) United States
(12) Patent Application Publication
(10) Pub. No.: US 2026/0151761 A1
(43) Pub. Date: Jun. 4, 2026

(54) METAL-ORGANIC FRAMEWORK HAVING TITANIUM AS METAL ELEMENT AND METHOD FOR PRODUCING THE SAME

(71) Applicant: CANON KABUSHIKI KAISHA, Tokyo (JP)

(72) Inventors: MASAAKI NAKABAYASHI, Kanagawa (JP); YU KAMENO, Kanagawa (JP)

(21) Appl. No.: 19/384,519

(22) Filed: Nov. 10, 2025

(30) Foreign Application Priority Data
Dec. 3, 2024 (JP) 2024-209891

(51) Int. Cl. B01J 31/16; B01J 35/39; B01J 37/00; B01J 37/04; B01J 37/08; C01B 3/04

(52) U.S. Cl. CPC B01J 31/1691; B01J 35/39; B01J 37/009; B01J 37/04; B01J 37/08; C01B 3/042

(57) ABSTRACT

A method for producing a metal-organic framework. The method includes: dissolving an acid compound having terephthalic acid as its main molecular structure, polyacrylic acid, and a silver-containing compound to produce an intermediate A; mixing a titanium-containing metal oxide cluster into the intermediate A to produce an intermediate B; heating the intermediate B to produce an intermediate C in a solid form; and repeatedly dissolving and centrifuging the intermediate C to produce a metal-organic framework.

### BACKGROUND

### Field of the Technology

The present disclosure relates to a metal-organic framework having titanium (Ti) as a metal element and a method for producing the same. The metal-organic framework is suitable for use as a photocatalyst to decompose water and produce hydrogen through introduction and promotion of a heterometallic compound.

### Description of the Related Art

Technology for using hydrogen as a clean energy source that does not produce carbon dioxide gas is significantly developing. Currently, there are three major methods for producing hydrogen: reacting hydrocarbon such as methane contained in petroleum and natural gas with steam to decompose it into hydrogen and carbon dioxide, steaming and burning coal to produce coal gas consisting of hydrogen and carbon monoxide, and passing an electric current through water to decompose it. However, these methods all have significant environmental and cost disadvantages, such as generation of carbon dioxide gas in the production step and consumption of large amounts of electricity to produce hydrogen by electrolysis. It is important to supply hydrogen in an environmentally friendly and low-cost manner for the future. To solve the above disadvantages, a photocatalytic reaction that uses solar energy to decompose water into hydrogen and oxygen is being researched and developed as a cutting-edge technology. Currently, metal oxides, such as barium titanate and strontium titanate, as photocatalysts achieved a high quantum efficiency.

A metal-organic framework (MOF), which is expected to have a surface area effect due to its microporous and dense structure and also holds great promise as a catalyst, is under active research and development. In a study of photocatalysis, a metal-organic framework having a titanium element cluster is also being studied as a photocatalyst. The current research challenge in using a metal-organic framework as a photocatalyst is to increase its catalytic efficiency, and study results have been published. For example, Yanghe Fu et al. at Fuzhou University disclosed that visible light absorption is enhanced by adding an amine to the linker constituting a metal-organic framework (Angewandte Chemie International Edition (IF16.6) Pub Date: 2012 Feb. 22), and Horiuchi et al. at Osaka Public University disclosed that optical absorption is increased by inducing structural defects (Journal of Catalysis 392(2020)119-125). In addition, Horiuchi et al. at Osaka Public University increased the efficiency by supporting an auxiliary catalyst within a metal-organic framework (Journal of Smart Processing vol. 2 No. 6 (2013 November) 287-292. Other examples of introducing an auxiliary catalyst include: synthesizing a metal-organic framework by coordinating a metal auxiliary catalyst to a linker (PCT Japanese Translation Patent Publication No. 2022-527963); adsorbing a nitrogen compound having an auxiliary catalyst onto a pre-made metal-organic framework (PCT Japanese Translation Patent Publication No. 2007-534651); and soaking a pre-made metal-organic framework in an inorganic salt solution to exchange with a cation (PCT Japanese Translation Patent Publication No. 2023-519686).

However, in the known technology described above, measures to increase catalytic efficiency are taken as a process after the production of the metal-organic framework. This causes disadvantages, such as an increase in the production cost due to the increased number of steps and lack of uniformity in the treated and added state. In addition, when a compound having an auxiliary catalyst is introduced so that the catalyst is supported, it is not easy to introduce the compound into the metal-organic framework in a solution, and the compound concentrates around the surface of the framework, resulting in a smaller high catalytic effect area. This does not allow the metal-organic framework to fully exhibit the inherent properties when used as a catalyst and also makes it difficult to use the metal-organic framework for, for example, a photocatalytic reaction that decomposes water into hydrogen and oxygen using solar energy.

### SUMMARY

In view of the above, the present disclosure is directed to a metal-organic framework that is capable of exhibiting enough catalytic efficiency as a photocatalyst that uses solar energy, and a method for producing the metal-organic framework that does not require post-processing and multiple steps.

The present disclosure relates to a metal-organic framework including a silver-containing compound inside the metal-organic framework, the metal-organic framework being an MIL-125-based metal-organic framework.

The present disclosure also relates to a method for producing a metal-organic framework, the method including: dissolving an acid compound having terephthalic acid as its main molecular structure, polyacrylic acid, and a silver-containing compound to produce an intermediate A; mixing a titanium-containing metal oxide cluster into the intermediate A to produce an intermediate B; heating the intermediate B to produce an intermediate C in a solid form; and repeatedly dissolving and centrifuging the intermediate C to produce a metal-organic framework.

Features of the present disclosure will become apparent from the following description of embodiments with reference to the attached drawings. The following description of embodiments is described by way of example.

### BRIEF DESCRIPTION OF THE DRAWINGS

FIG. 1 is a flowchart schematically indicating a method for producing a metal-organic framework according to the present disclosure.

FIG. 2 shows results of SEM observation of metal-organic frameworks synthesized by adding only polyacrylic acid.

FIG. 3 is a graph showing results of X-ray diffraction measurements of a regular synthetic product, a polyacrylic acid added synthetic product, and a metal-organic framework according to the present disclosure.

FIG. 4 is a graph showing results of X-ray diffraction measurements of samples of Comparative Examples.

FIG. 5 shows compositions and observation results of Examples.

FIG. 6 is a graph showing results of X-ray diffraction measurements of Examples.

FIG. 7 is a graph showing a comparison between Examples and Comparative Examples in terms of background intensity value/peak intensity value (Ib/I).

### DESCRIPTION OF THE EMBODIMENTS

### Metal-Organic Framework (MOF)

Titanium-based metal-organic framework (Ti-MOF) is part of the MOF family, and MIL-125 is a Ti-based MOF derived from Ti metal ions and organic ligands.

The metal-organic framework of this disclosure is an MIL-125-based metal-organic framework, wherein a silver-containing compound is present inside the metal-organic framework.

The metal-organic framework according to the present disclosure has a silver-containing compound serving as an auxiliary catalyst in the framework of the metal-organic framework and thus has higher catalytic effect, and the metal-organic framework as a photocatalyst is expected to decompose water more efficiently than known photocatalysts.

The MIL-125-based metal-organic framework can be synthesized from an organic linker and a titanium-containing metal oxide cluster.

The organic linker is an acid compound having terephthalic acid as its main molecular structure. An acid compound having terephthalic acid as its main molecular structure may be a terephthalic acid derivative. The terephthalic acid derivative may have an amino group to increase optical absorption in the visible region, and the terephthalic acid derivative having an amino group may be 2-amino-terephthalic acid and its derivatives.

The titanium-containing metal oxide cluster is not limited. Examples of organic titanate, which is a titanium-containing metal oxide cluster, include titanium (IV) isopropoxide, titanium (IV) butoxide, and tetrabutyl orthotitanate. Among them, tetrabutyl orthotitanate can be particularly used as the titanium-containing metal oxide cluster.

The silver-containing compound is not limited. Examples of the silver-containing compound include silver benzoate, silver carbonate, silver chloride, silver bromide, and silver acetate. Among them, silver benzoate can be particularly used as the silver-containing compound.

The metal-organic framework according to the present disclosure may contain polyacrylic acid, and the presence of polyacrylic acid can induce defects in the structure of the metal-organic framework, as will be described in detail in the sections of Method for Producing Metal-Organic Framework and Examples.

In the present disclosure, the silver-containing compound serving as an auxiliary catalyst is present “inside” the framework of the metal-organic framework because the method for producing a metal-organic framework according to the present disclosure induces and increases defects in the structure of the metal-organic framework, allowing the introduction and promotion of a heterometallic compound (silver-containing compound) serving as an auxiliary catalyst into voids formed by these defects. In a 2θ-θ X-ray diffraction measurement of a powder of the produced metal-organic framework using a wavelength of 1.5418 Å, when a ratio (Ib/I) of a background intensity (Ib) to a peak intensity (I) at 2θ=6.74 degrees is 0.1 or more, voids sufficient to allow the silver-containing compound to be present inside the framework of the metal-organic framework are generated by the defects in the metal-organic framework. The ratio of Ib/I is preferably 0.3 or more, and more preferably 0.5 or more.

### Method for Producing Metal-Organic Framework

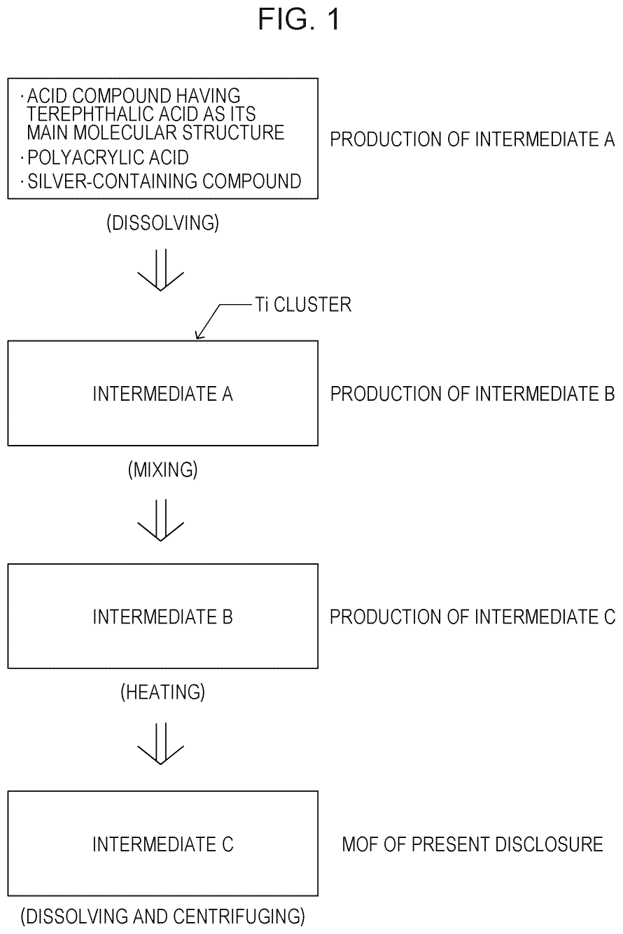

FIG. 1 is a flowchart schematically indicating a method for producing a metal-organic framework according to the present disclosure.

First, in the step of producing an intermediate A, an acid compound having terephthalic acid as its main molecular structure, polyacrylic acid, and a silver-containing compound, which are the above-described constituent components, are dissolved to produce the intermediate A.

Specifically, 2-aminoterephthalic acid, as an acid compound having terephthalic acid as the main molecular structure, is dissolved in a dimethylformamide solvent, and then polyacrylic acid and silver benzoate are mixed. This mixed solution is mixed with a mixed solution of a methanol solvent and silver benzoate in a nitrogen purged glove box and stirred thoroughly to produce the intermediate A.

Here, it is necessary to dissolve and mix these components simultaneously. This allows polyacrylic acid to induce a disruption of the structure of the MIL-125-based metal-organic framework and also allows the silver-containing compound to increase the structural defects, allowing the silver-containing compound to be present inside the metal-organic framework.

Next, into the intermediate A, the above-described titanium-containing metal oxide cluster, such as titanium isopropoxide, is mixed to produce an intermediate B.

Furthermore, the intermediate B is put into an autoclave and heated to produce a solid intermediate C, and the produced intermediate C is repeatedly subjected to dissolution and centrifugation to produce the desired metal-organic framework.

The production steps of the method for producing the metal-organic framework according to the present disclosure are as described above. The following describes a specific example of “a method for producing a Ti-cluster-based metal-organic framework”. Metal-organic frameworks produced by “a method for producing a metal-organic framework synthesized by adding only polyacrylic acid” and “a method for producing a metal-organic framework according to the present disclosure”, which are based on the specific example, were subjected to structural analysis by SEM observation and X-ray diffraction.

### Method for Producing Ti-Cluster-Based Metal-Organic Framework

The production method described in M. A. Nasalevich et al. was used. The metal-organic framework used in this proposal is one labeled MIL-125(Ti)-NH₂. The production method is as follows. An amino group is optional.
(1) Dissolve 2.86 g (15.8 mol) of 2-aminoterephthalic acid in a mixture of 40 mL of dimethylformamide (DMF) and 10 mL of dry methanol (MEOH).
(2) Then, add 2.86 mL (9.7 mol) of titanium isopropoxide to the mixture and put it into an autoclave.
(3) Seal the autoclave and heat the mixture at 110° C. for 72 hours.
(4) Filter the yellow product, wash the product with fresh dimethylformamide, and stir the product all day and night with 50 mL of dimethylformamide per 1 g of the product. This removes excess linker.
(5) Repeat (4) twice by using methanol to replace dimethylformamide with methanol.
(6) Dry the product at 100° C.

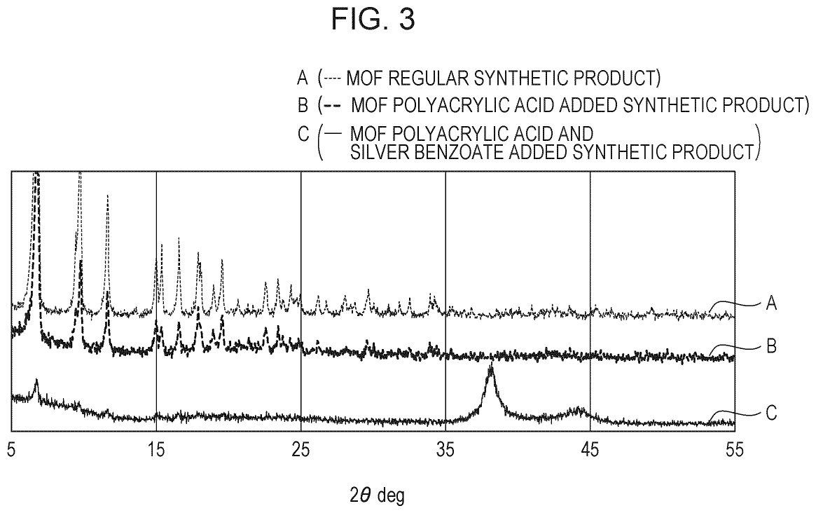

FIG. 3 (line A in FIG. 3) shows the result of structural analysis by X-ray diffraction of the dried product (MOF regular synthetic product). The line A shows a peak similar to that reported in Electronic Origins of Photocatalytic Activity in d0 Metal Organic Frameworks, Sci. Rep. 6(2016)23676, indicating that the MOF structure is formed.

Method for Producing Metal-Organic Framework Synthesized by Adding Only Polyacrylic Acid

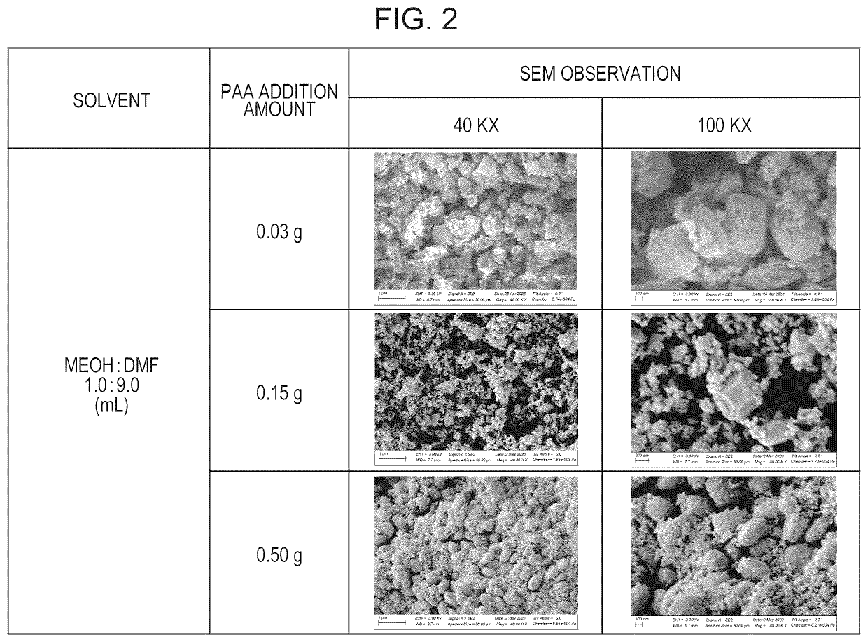

The following describes a case in which polyacrylic acid (PAA) is added in the step (1) above. FIG. 2 shows SEM images of the metal-organic frameworks produced by adding polyacrylic acid (molecular weight 5000, Wako Pure Chemical Corporation) to solvents at concentrations of 0.6 g/L, 3.0 g/L, and 9.0 g/L. It is found that the presence of polyacrylic acid changes the shape of the metal-organic framework from a partial octahedral shape to a cocoon-like shape as the amount of the polyacrylic acid increases.

FIG. 3 (line B in FIG. 3) shows the result of structural analysis by X-ray diffraction of the case in which the polyacrylic acid was added at a concentration of 9.0 g/L. It shows that the peak intensity of the sample having a cocoon-like shape is weaker than that of the above regular synthetic product of MOF, but the sample still shows the characteristic reflection peak of MIL-125(Ti)-NH₂ and generally keeps the dense structure of the metal-organic framework.

### Method for Producing Metal-Organic Framework According to Present Disclosure

In the production method according to the present disclosure, polyacrylic acid and silver benzoate are dissolved and mixed together with 2-aminoterephthalic acid in the step (1) of the above-described method for producing a Ti-cluster-based metal-organic framework, and then the step (2) and the following steps are performed. Polyacrylic acid is added at a concentration of 8.57 g/L to the solvent (the amount substantially equivalent to that in the above example in which polyacrylic acid was added at a concentration of 9.0 g/L), and silver benzoate (Tokyo Chemical Industry Co., Ltd.) is added at a 20 mol % relative to the 2-aminoterephthalic acid.

The line C in FIG. 3 shows the result of the analysis by X-ray diffraction of the produced sample. As mentioned earlier, polyacrylic acid induces a disruption of the structure of the MIL-125-based metal-organic framework, and the presence of the structural defects in the MIL-125-based metal-organic framework induced by polyacrylic acid makes it easier for the silver-containing compound to be present inside the metal-organic framework. However, in this production method, the characteristic reflection peak of MIL-125(Ti)-NH₂ has significantly weakened. This indicates that the addition of silver benzoate together with polyacrylic acid increased the structural defects even more. In addition, the background is larger at diffraction angles (2θ) of 10 degrees or less, indicating scattering by the structure has increased.

### EXAMPLES

The present disclosure will be described in further detail by using the following Comparative Examples (control) and Examples.

### COMPARATIVE EXAMPLES

MIL-125(Ti)-NH₂ is examined without additives (polyacrylic acid and silver benzoate) to see the influence of the mixing ratio of the base materials and solvent. Table 1 shows the composition. In Table 1, Linker is 2-aminoterephthalic acid (Tokyo Chemical Industry Co., Ltd.), Metal Oxide Cluster is tetrabutyl orthotitanate (Tokyo Chemical Industry Co., Ltd.), Solvent 1 is dimethylformamide (Tokyo Chemical Industry Co., Ltd.), and Solvent 2 is dry methanol (Hayashi Pure Chemical Ind., Ltd.). The “standard recipe” follows the regular synthetic product of the above MOF.

TABLE 1

| | Standard Recipe | | Experiment 1 | | Experiment 2 | | Experiment 3 | | Experiment 4 | |
|---|---|---|---|---|---|---|---|---|---|---|
| Linker | 15.8 | mol | 15.8 | mol | 31.6 | mol | 15.8 | mol | 15.8 | mol |
| Metal Oxide | 9.7 | mol | 18.6 | mol | 9.7 | mol | 9.7 | mol | 9.7 | mol |
| Cluster | | | | | | | | | | |
| Solvent 1 | 40 | ml | 40 | ml | 60 | ml | 40 | ml | 80 | ml |
| Solvent 2 | 10 | ml | 10 | ml | 10 | ml | 20 | ml | 10 | ml |

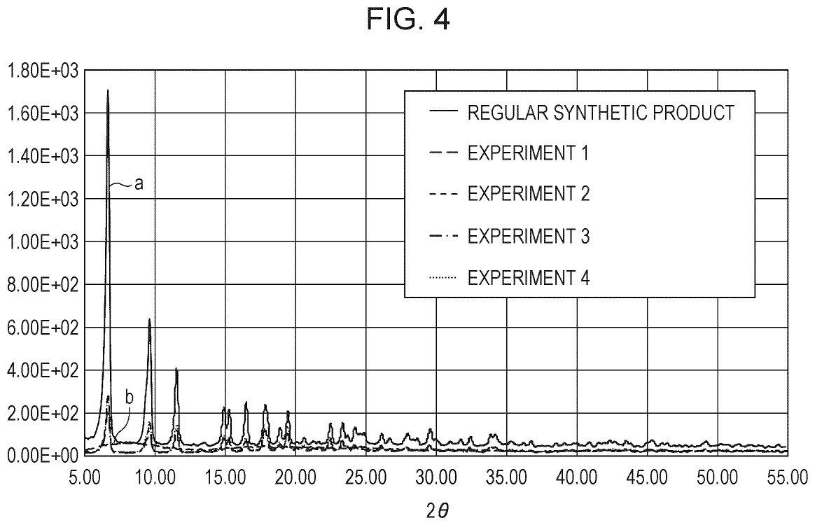

FIG. 4 shows the X-ray diffraction results of samples in Table 1 having different mixing ratios. The X-ray diffraction results in FIG. 4 show that the background does not increase (Ib/I does not change significantly) simply by changing the mixing ratio of the basic materials and solvent. When the amount of the metal cluster was doubled as in Experiment 1, no synthesis was established and no reflection peak appeared at all (line “b” in FIG. 4). The regular synthetic product (line “a” in FIG. 4) has a high yield of MIL-125(Ti)-NH₂, and the other products (Experiments 2, 3, and 4), which have largely different compositions from the standard, has a low yield of MIL-125(Ti)-NH₂ and thus differ from the regular synthetic product in the magnitude of the background.

### Examples 1 to 5

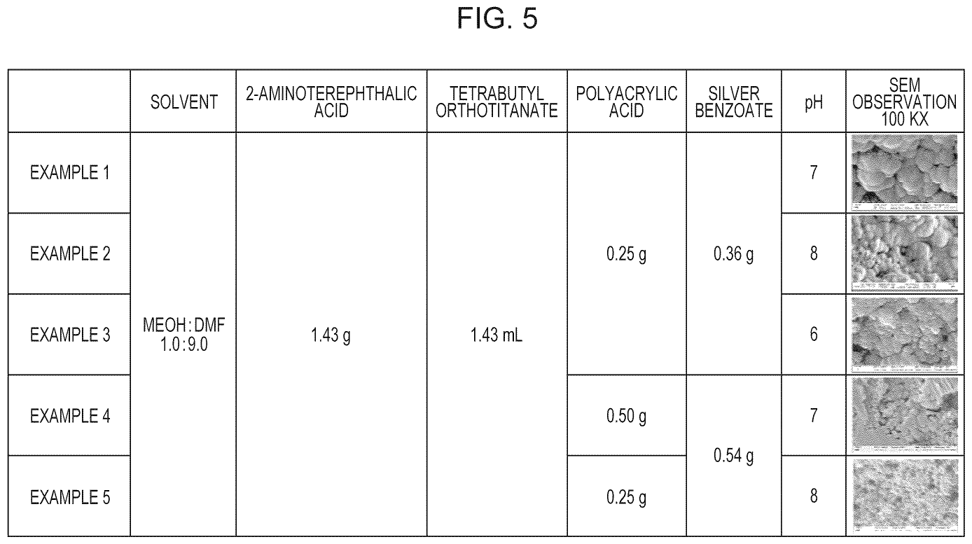

The synthesis of the metal-organic framework having polyacrylic acid and silver benzoate was carried out to produce samples of Examples 1 to 5 using the compositions shown in FIG. 5. The pH value was adjusted because it is known that the octahedrality of the particles of MIL-125(Ti)-NH₂ is affected by the pH value of the synthesis solution.

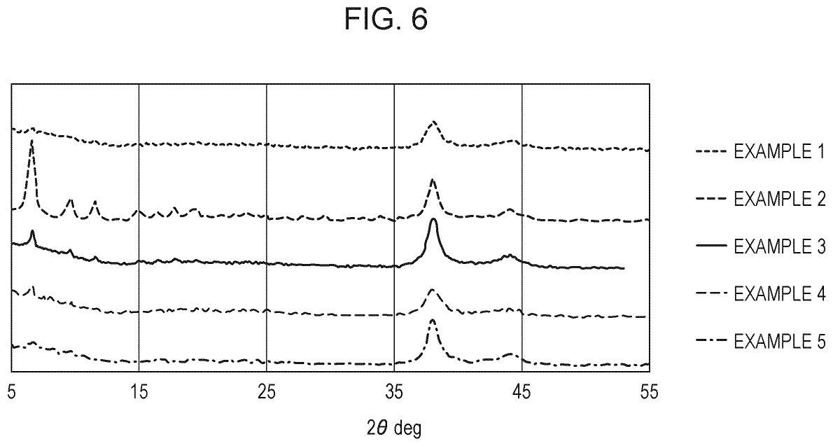

The results are shown in FIGS. 5 and 6. FIG. 5 shows the results of SEM observations on cross-sectioned samples encapsulated in resin, as well as the compositions and pH values. FIG. 6 shows the results of reflection measurements by using an X-ray diffractometer. All products differ in the reflection peak intensity specific to MIL-125(Ti)-NH₂, indicating that the degree to which the original structure is maintained within the structure of the metal-organic framework varies.

Here, the results of Comparative Examples and Examples 1 to 5 are compared in terms of background intensity (Ib)/peak intensity (I) at a diffraction angle of 6.74 degrees that produces a large reflection peak. As an X-ray diffractometer, Ultima IV available from Rigaku Holdings Corporation was used. The diffraction pattern was obtained by a 2θ-θ measurement using this X-ray diffractometer. The tube was a Cu tube, and the measurement wavelength λ was 1.5418 Å. The tube voltage was 40 kV, and the tube current was 40 mA. The temperature was 23° C.±2° C. The range of 20 was from 5° to 55°, the sampling width was 0.02°, and the speed was 2°/min (two accumulations). The slits were as follows: outgoing and incoming solar slit, 5°; diverge slit, ¼; height limiting slit, 10 mm; receiving slit, open; and scattering slit, ⅔. The background values were calculated using the values obtained by analysis performed using Data Analysis Software PDXL2 accompanying the diffractometer.

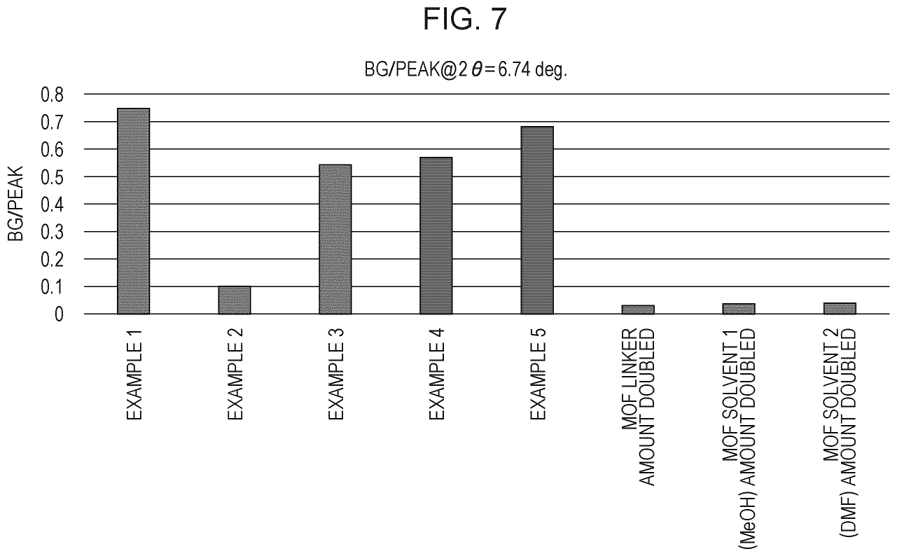

The results of the comparison are shown in FIG. 7, where the vertical axis “BG/PEAK” is background intensity (Ib)/peak intensity (I), “MOF Linker Amount Doubled” corresponds to Experiment 2 of Comparative Example, “MOF Solvent 1 Amount Doubled” corresponds to Experiment 4 of Comparative Example, and “MOF Solvent 2 Amount Doubled” corresponds to Experiment 3 of Comparative Example. FIG. 7 does not show the result of Experiment 1 of Comparative Example in which the synthesis was not established.

The results shown in FIG. 7 indicate that there is a clear difference in the values between the samples of Comparative Examples, which do not contain polyacrylic acid and silver benzoate and have different mixing ratios of the basic materials, and the samples of Examples, which contain polyacrylic acid and silver benzoate. It was confirmed, from the data of Examples 1 to 5 in FIG. 7, especially the data of Example 2, that it can be said that when the ratio (Ib/I) of the background intensity (Ib) to the peak intensity (I) at 2θ=6.74 degrees under the above measurement conditions is 0.1 or more, the structural anisotropy increased and voids sufficient to allow a silver-containing compound to be present inside the framework of the metal-organic framework were formed by defects in the metal-organic framework.

### Configurations Included in Embodiments of Present Disclosure

The disclosure of the embodiments includes the following configurations.

(Configuration 1) A metal-organic framework including a silver-containing compound inside the metal-organic framework, the metal-organic framework being an MIL-125-based metal-organic framework.

(Configuration 2) The metal-organic framework according to Configuration 1, wherein, in a 2θ-θ X-ray diffraction measurement of a powder of the metal-organic framework using a wavelength of 1.5418 Å, a ratio (Ib/I) of a background intensity (Ib) to a peak intensity (I) at 2θ=6.74 degrees is 0.1 or more.

(Configuration 3) The metal-organic framework according to Configuration 1 or 2, wherein the metal-organic framework contains polyacrylic acid.

(Configuration 4) The metal-organic framework according to any one of Configurations 1 to 3, wherein the silver-containing compound is silver benzoate.

(Configuration 5) The metal-organic framework according to any one of Configurations 1 to 4, wherein the metal-organic framework is used in a method that uses the metal-organic framework as a photocatalyst to decompose water and produce oxygen and/or hydrogen.

(Configuration 6) A method for producing a metal-organic framework, includes dissolving an acid compound having terephthalic acid as its main molecular structure, polyacrylic acid, and a silver-containing compound to produce an intermediate A; mixing a titanium-containing metal oxide cluster into the intermediate A to produce an intermediate B; heating the intermediate B to produce an intermediate C in a solid form; and repeatedly dissolving and centrifuging the intermediate C to produce a metal-organic framework.

(Configuration 7) The method for producing a metal-organic framework according to Configuration 6, wherein, in the production of the intermediate A, the acid compound having terephthalic acid as its main molecular structure, the polyacrylic acid, and the silver-containing compound are dissolved simultaneously.

(Configuration 8) The method for producing a metal-organic framework according to Configuration 6 or 7, wherein the acid compound having terephthalic acid as its main molecular structure has an amino group.

(Configuration 9) The method for producing a metal-organic framework according to any one of Configurations 6 to 8, wherein the silver-containing compound is silver benzoate.

(Configuration 10) The method for producing a metal-organic framework according to any one of Configurations 6 to 9, wherein the titanium-containing metal oxide cluster is tetrabutyl orthotitanate.

(Configuration 11) The method for producing a metal-organic framework according to any one of Configurations 6 to 10, wherein the silver-containing compound is present inside the produced metal-organic framework.

(Configuration 12) The method for producing a metal-organic framework according to any one of Configurations 6 to 11, wherein, in a 2θ-θ X-ray diffraction measurement of a powder of the produced metal-organic framework using a wavelength of 1.5418 Å, a ratio (Ib/I) of a background intensity (Ib) to a peak intensity (I) at 2θ=6.74 degrees is 0.1 or more.

Since the method for producing a metal-organic framework according to the present disclosure can uniformly introduce a silver compound as an auxiliary catalyst into the metal-organic framework, the produced metal-organic framework can be suitably used in the method for producing hydrogen, which is attracting attention as a clean energy source, in which water is decomposed into hydrogen and oxygen by using a photocatalyst using solar energy and in a hydrogen production apparatus including a component having a metal-organic framework.

According to the present disclosure, structural defects are efficiently increased in the titanium-based metal-organic framework, and a silver compound as an auxiliary catalyst is incorporated in the process of synthesizing the metal-organic framework. This enables the auxiliary catalyst to be uniformly introduced into the metal-organic framework.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

This application claims the benefit of Japanese Patent Application No. 2024-209891, filed Dec. 3, 2024, which is hereby incorporated by reference herein in its entirety.

What is claimed is:

1. A metal-organic framework comprising a silver-containing compound inside the metal-organic framework, the metal-organic framework being an MIL-125-based metal-organic framework.

2. The metal-organic framework according to claim 1, wherein, in a 2θ-θ X-ray diffraction measurement of a powder of the metal-organic framework using a wavelength of 1.5418 Å, a ratio (Ib/I) of a background intensity (Ib) to a peak intensity (I) at 2θ=6.74 degrees is 0.1 or more.

3. The metal-organic framework according to claim 1, wherein the metal-organic framework contains polyacrylic acid.

4. The metal-organic framework according to claim 1, wherein the silver-containing compound is silver benzoate.

5. The metal-organic framework according to claim 1, wherein the metal-organic framework is used in a method that uses the metal-organic framework as a photocatalyst to decompose water and produce oxygen and/or hydrogen.

6. A method for producing a metal-organic framework, comprising:
dissolving an acid compound having terephthalic acid as its main molecular structure, polyacrylic acid, and a silver-containing compound to produce an intermediate A;
mixing a titanium-containing metal oxide cluster into the intermediate A to produce an intermediate B;
heating the intermediate B to produce an intermediate C in a solid form; and
repeatedly dissolving and centrifuging the intermediate C to produce a metal-organic framework.

7. The method for producing a metal-organic framework according to claim 6, wherein, in the production of the intermediate A, the acid compound having terephthalic acid as its main molecular structure, the polyacrylic acid, and the silver-containing compound are dissolved simultaneously.

8. The method for producing a metal-organic framework according to claim 6, wherein the acid compound having terephthalic acid as its main molecular structure has an amino group.

9. The method for producing a metal-organic framework according to claim 6, wherein the silver-containing compound is silver benzoate.

10. The method for producing a metal-organic framework according to claim 6, wherein the titanium-containing metal oxide cluster is tetrabutyl orthotitanate.

11. The method for producing a metal-organic framework according to claim 6, wherein the silver-containing compound is present inside the produced metal-organic framework.

12. The method for producing a metal-organic framework according to claim 6, wherein, in a 2θ-θ X-ray diffraction measurement of a powder of the produced metal-organic framework using a wavelength of 1.5418 Å, a ratio (Ib/I) of a background intensity (Ib) to a peak intensity (I) at 2θ=6.74 degrees is 0.1 or more.

13. An apparatus for producing hydrogen, comprising:
a component; and
a metal-organic framework provided in the component, wherein
the metal-organic framework includes a silver-containing compound inside the metal-organic framework and is an MIL-125-based metal-organic framework.
